(19) United States
(12) Patent Application Publication
(10) Pub. No.: US 2010/0084453 A1
(43) Pub. Date: Apr. 8, 2010

(54) Circular stapler

(75) Inventor: Ren-Min Hu, Changzhou (CN)

Correspondence Address: Ren-Min Hu, P.O. Box 166-13, Taipei, Taiwan R.O.C., Taipei, Taiwan, 115, TW

(73) Assignee: Changzhou Waston Medical Appliance Co., Ltd

(21) Appl. No.: 12/285,378

(22) Filed: Oct. 3, 2008

(51) Int. Cl. A61B 17/064

(52) U.S. Cl. 227/179.1

(57) ABSTRACT

A circular stapler facilitate performing end to end anastomosis; further an occasional prompt with sound effect warning timely firing the stapler bolts. A circular stapler includes a bolting machine (1) consists of a shaft (11), an actuator (12), and a stapler (13); a hand-held handle (2); an adjusting knob (3); and an anvil (4), a hollow anvil shaft (41);
characterized in that: a conic trocar (5) disposed to a most distal end of said bolting machine (1), a cambered surface of said conic trocar (5) formed with, at least, three relieve-pressure grooves (53) equal distant distributed are extended from an apex (51) to a bottom (52); an audio prompt (6) disposed inside said bolting machine (1), said audio prompt (6) includes a printed circuit board (PCB) (61), a buzzer (62) electrically connected to the PCB (61) controlled by a switch, and a battery (64) supplies power to the PCB (61).

### FIELD OF THE INVENTION

The present invention is related to a circular stapler; particularly on thoracic surgery, slimming surgery, colectomy and proctectomy for end-to-end, end-to-side and side-to-side anastomoses.

### DESCRIPTION OF PRIOR ART

For decades, to abridge operation time for surgery scheduling, ease post-operation pain, circular stapler adopted as a less invaded procedure, but an efficient instrument operated within a short mending and seaming time, during surgical operations. With fired bolts, said circular stapler performing on internal body tissue is acknowledged as a better procedure applied to some surgery operations. For instance, stapler invaded through anus to remove prolapse of internal hemorroids was labeled “Procedure for Prolapse and Hemorroids (PPH)”, first adopted by Dr. Antonio Longo in 1993 and promoted in 1998. To meet more and more surgical removal and mending and seaming requirements finished in a few steps, said circular stapler have been developed and furnished with bolts for those surgery operations in practice.

Anastomosis is a surgical procedure to reconnect two ends of two tubular tissues to form a straight passage. Before the circular stapler is introduced, said end to end anastomosis procedure was performed with a lot steps through a complicated and time-consuming process. Surgeon had to remove infected portions exactly; allowed an incised tract end to intestine's end be sure to work stitch by stitch, at the same time, said incised tract end to be reconnected must be in alignment with the stitched intestine's end.

With lower tract end to end anastomosis developed into a stapler and bolts carrying a full-promised reconnection, a lot of steps in the prior arts can be simplified to only a few steps of the introduced procedure further to abridge operation time for surgery scheduling.

Conventional circular stapler usually includes a long shaft, an actuator disposed to a proximal end of the shaft and a stapler disposed to a distal end of the shaft. Said stapler usually includes a cartridge fixture with a plurality of bolts axially arranged in a concentric array; a circular blade rotatably mounted inside said cartridge fixture behind the bolts. In operation, said circular blade is axially moved to said distal end. A conventional trocar shaft is axially extended from a center of said cartridge fixture. Said trocar shaft can be axially moved relative to said cartridge fixture. An anvil disposed to said trocar shaft with a conventional bolt anvil face installed thereto accommodates said bolts readily to be fired to reconnect end of incised tract and intestine's end. A distal end of said cartridge fixture kept a distance to said bolt anvil face can be controlled by an adjustment mechanism disposed to a proximal end of said trocar shaft. Internal body tissue received between said cartridge fixture and said bolt anvil face can be removed and stapled at the same time, when the surgeon activate said actuator.

As shown in FIG. 1, a perspective view of said conventional trocar is illustrated. A trocar (10) is mounted to a most distal end of said trocar shaft fixed to the anvil.

When a conventional stapler performed end to end anastomosis, after removal of trial sample, an anvil of said stapler inserted into a proximal end of the incised tract adjacent to a staple line. Such an end to end anastomosis is subject to whether the anvil is inserted through an incised opening of said incised tract. Sometimes, said anvil is disposed to a distal end of said bolt, and then inserted said stapler through rectum and anus to move said anvil in place. Then the surgeon seamed a stitch to tie a proximal end of said intestine to a shaft of said anvil. After that, said infected portion tied up around said stitch can be removed. With said trocar, the surgeon can fix said anvil to said trocar shaft of said stapler of in tract anastomosis.

And then, a gap between said anvil and said cartridge fixture is reduced by said surgeon, whereby end of said incised tract and said intestine's end can be reconnected within said reduced gap. Finally, the in tract stapler is activated by said surgeon, arrays of bolts can be driven through both end of said incised tract, and said intestine's end, which is reconnected to form a straight passage. Meanwhile, as said bolts are driven and applied to reconnection, said circular blade is driven through said intestine tissue to remove said infected portion from between said ends. The surgeon pulled out said stapler to finish said end to end anastomosis.

Said conventional trocar (10) has some drawbacks as following:

1. Surgeon fixed said anvil to said trocar shaft, both faces of them were complete in contact, and conformed to each other. Or resistance and pressure caused between faces in interference fit may disrupt procedure of operation.

2. Through end to end anastomosis, the surgeon activated said in tract stapler must have seen through “firing indicator window” or “firing indicator scale” for ascertaining operation of said stapler in time. It may result in earlier or later firing than the opportune time so that the procedure of operation with precision must be prolonged.

### SUMMARY OF THE INVENTION

Accordingly, the present invention is aimed to provide a circular stapler to relieve a surgeon's pressure and facilitate performing end to end anastomosis; further an occasional prompt with sound effect warning the surgeon timely firing the stapler bolts.

A circular stapler includes a bolting machine (1) consists of a shaft (11), an actuator (12) disposed to a proximal end of said shaft (11) and a stapler (13) disposed to a distal end of said shaft (11);

a hand-held handle (2) disposed to a bottom side of said actuator (12), when a safety catch (21) is released, operation of said handle can fire bolts from the stapler;

an adjusting knob (3) disposed to a proximal end of said actuator (12); and

an anvil (4) is connectable to said stapler (13), a hollow anvil shaft (41) is disposed to a proximal end of said anvil (4);

characterized in that:

a conic trocar (5) disposed to a most distal end of said bolting machine (1), said trocar (5) fit in said anvil shaft (41); a cambered surface of said conic trocar (5) formed with, at least, three relieve-pressure grooves (53) equal distant distributed are extended from an apex (51) to a bottom (52);

An audio prompt (6) is disposed inside said bolting machine (1), said audio prompt (6) includes a printed circuit board (PCB) (61), a buzzer (62) electrically connected to the PCB (61), a switch (63) switches on/off said buzzer (62) and a battery (64) supplies power to the PCB (61).

Said circular stapler as claimed in claim 1 wherein an included angle between each two adjacent relieve-pressure grooves (53) converged at the apex is 120 degrees.

Said circular stapler as claimed in claim 1 wherein said relieve-pressure grooves (53) are concaved in said cylindrical surface and cambered in shape.

Said circular stapler as claimed in claim 1 wherein a switch (63) disposed to a lateral face near said distal end of said bolting machine (1), where a firing indicator window showing a firing indicator scale formed (not shown).

Said circular stapler as claimed in claim 1 wherein a proximal end of said anvil shaft (41) is formed with a clip notch (42).

Said circular stapler as claimed in claim 1 wherein said safety catch (21) disposed between said bolting machine (1) and said handle (2) can be used to ensure an opportune firing of bolts.

### ADVANTAGES OF EMBODIMENTS OF THE INVENTION

Advantages and benefits can be achieved by embodiments of the present invention as following:
1. Three relieve-pressure grooves (53) equal distant distributed on the cylindrical surface of said trocar are extended from said apex (51) to the bottom (52) are in contact with said anvil (4). When said anvil is fixed to said trocar shaft, either said trocar is in contact with said anvil (4), or said trocar is penetrated through internal body tissues, the contacted portions of said torcar can be reduced. Thereby, resistance and pressure can be reduced.

A sound prompt (6) is designed for a gap between end of incised tract and intestine's end to be reconnected is reduced, and for example, prolapse of internal hemorroids is removing, the buzzer (62) emits sound, to indicate that it's time to fire bolts from the in tract stapler without looking the firing indicator scale (not shown) of firing indicator window (not shown), earlier or later than the opportune firing can be avoided. The surgeon will have a tactile and on audible indication that the stapler has been fired.

### BRIEF DESCRIPTION OF DRAWINGS

FIG. 1 is a perspective view of the trocar of prior art.

FIG. 2 is a perspective view of the trocar of the present invention.

FIG. 3 is a front view of the trocar of the present invention.

FIG. 4 is a schematic view of the present invention.

FIG. 5 is a wiring diagram of the present invention.

FIG. 6 is a schematic view of the trocar in practice for an end to end anastomosis.

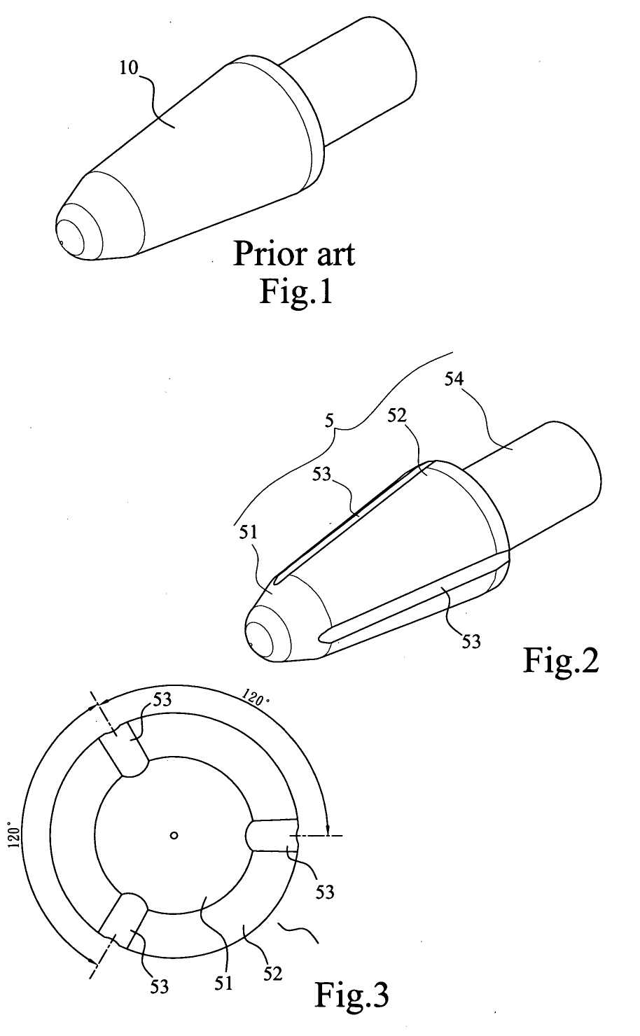

### DETAIL DESCRIPTION OF PREFERRED EMBODIMENT

The description is described in detail according the appended drawing hereinafter.

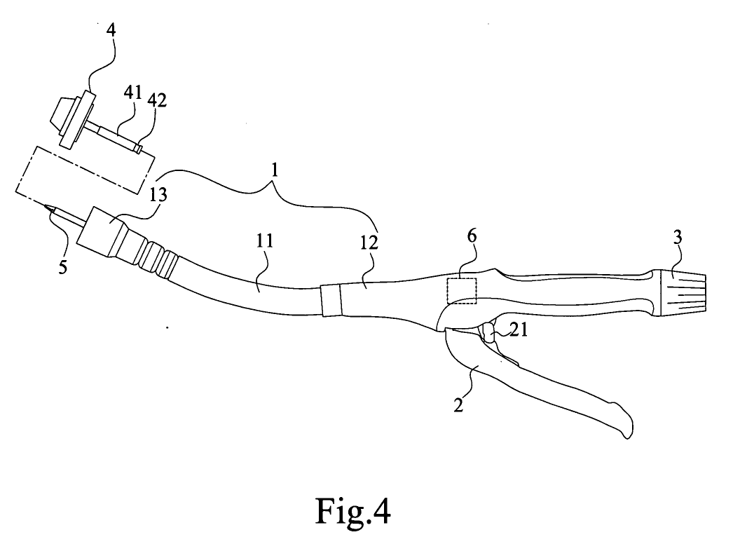

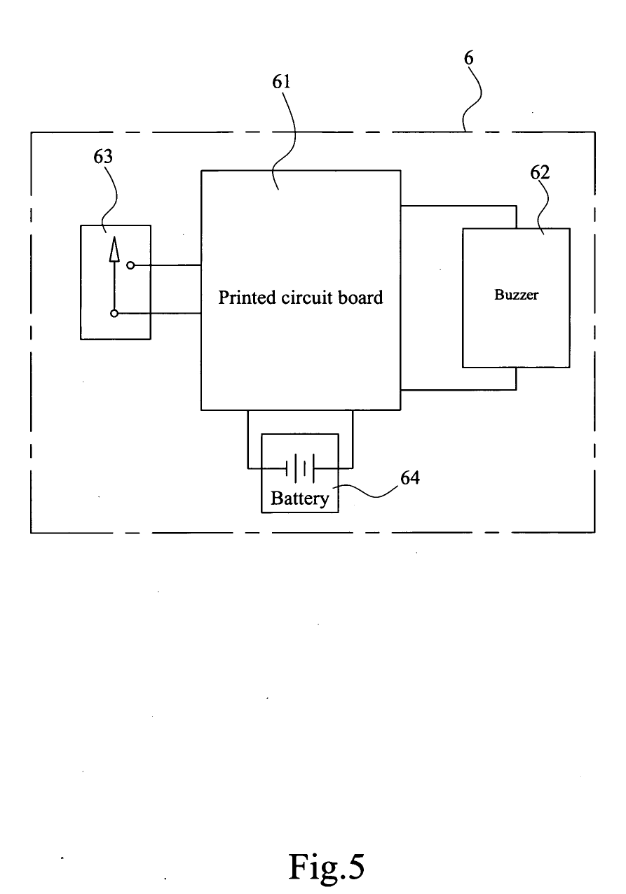

As shown in FIG. 2-5, a circular stapler includes a bolting machine (1) includes a long shaft (11), an actuator (12) disposed to a proximal end of said shaft (11) and a stapler (13) disposed to a distal end of said shaft (11); a hand-held handle (2) is a fork branched handle extended below a bottom side of said actuator (12), when a safety catch (21) is released, said handle is operated to fire bolts from the stapler;

An adjusting knob (3) disposed to a proximal end of said actuator (12); and an anvil (4) connected to said stapler (13), a hollow anvil shaft (41) is disposed to a distal end of said anvil (4).

Said circular stapler is emphasized by a conic trocar (5) fit in the anvil shaft (41), is disposed to a most distal end of the bolting machine (1), at least three relieve-pressure grooves (53) are formed from an apex (51) to a bottom (52) on a cylindrical surface; said three grooves (53) are concaved in the cylindrical surface and cambered in shape.

A sound prompt (6) disposed to said bolting machine (1), said sound prompt (6) includes a printed circuit board (PCB) (61), a buzzer (62) electrically connected to the PCB (61), a switch (63) switches on/off a buzzer (62) and a battery (64) supplies power to the PCB (61).

Said firing handle (2) is operated to fire bolts from said circular stapler. Said firing handle (2) is activated after the safety catch (21) is released. A trigger (not shown) inside the shaft (11) can force said bolts ejected from the stapler (13). Said bolts are ejected along a circumference of said anvil to be bent along said anvil's circumference.

Meanwhile, a circular blade fixed inside the stapler (13) is used to cut off infected portion stably trapped within a circumference of said stapler (13), and the infected portion clapped over a reduced gap between said anvil (4) and said stapler (13). Afterwards the circular stapler is pulled out or retracted through the tract, reconnected end of incised tract and intestine's end formed as a straight passage in position.

Furthermore, since a cylindrical surface of said trocar (5) is formed with, at least, three relieve-pressure grooves (53) extended from an apex (51) to a bottom (52). Those three relieve-pressure grooves are equally distant arranged on the trocar (5). Thus, an overall surface area of said cylindrical surface of said trocar is reduced to an extent that the trocar (5) goes through tissues with less pressure and resistance. Therefore, end of incised tract and intestine's end can be reconnected promptly.

Said sound prompt (6) readily disposed to said bolting machine is activated, whenever a surgeon moves said in tract stapler to reconnect end of incised tract to intestine end, which are trapped and clapped over within a reduced gap about said circumference of the stapler, an opportune firing time warning is promptly issued by said buzzer. The surgeon can therefore exactly fire the bolts to reconnect the incised tract and the intestine to form a straight passage. Earlier or later than the opportune time firing bolts may prolong the operation procedure.

Since the buzzer is exactly designed associated with a “fire” command to facilitate the firing handle (2) activation preferably but not necessarily seeing the firing indicator scale (not shown) through firing indicator window (not shown). Audible firing indicator catches surgeon's attention in advance of kinds of “seeing through” indicators, the surgeon can fire the bolts with precision.

An included angle between each two adjacent relieve-pressure grooves (53), which are converged at the apex and extended to the bottom of the trocar's cylindrical surface, is equally divided to 120 degrees. Said grooves (53) separated from one another by such a desired angle may reduce most pressure and resistance encountered by the tissues to be reconnected. And a symmetrical distribution of said grooves designed with an equation of force balance may facilitate the surgery operation performed sooner and better. Said relieve-pressure grooves (53) are concaved in the cylindrical surface of the trocar and each groove is cambered in shape. Resistance and pressure is reduced in symmetry, and the trocar (5) rigidity is established with balance grooves easy to manufacture.

A switch (63) is disposed to a lateral face near said distal end of said bolting machine (1), where a firing indicator window showing a firing indicator scale formed (not shown), the switch is operated in view of the sound prompt (6) already gave a “fire” command, the surgeon may check whether the bolt is ejected in position.

A clip notch (42) is disposed to a proximal end of said anvil shaft (41), said trocar (5) is joined together with the anvil shaft (41) by a snug fit between the clip notch (42) and the trocar (5).

A safety catch (21) disposed between said bolting machine (1) and firing handle (2) to prevent any accidental firing. Or the surgeon performs end to end anastomosis may fire the bolts from the in tract stapler wrongly by forcibly exerted force against his will.

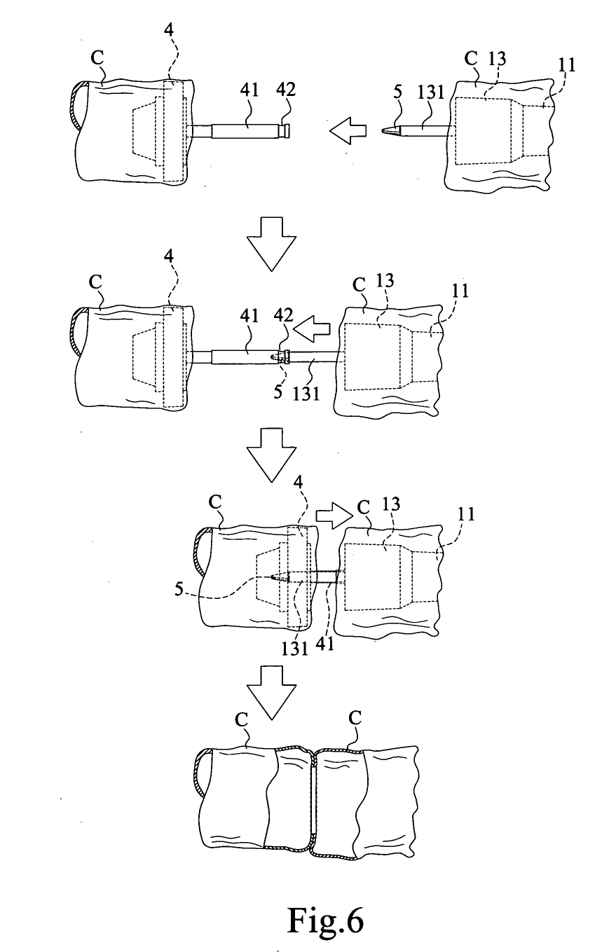

As shown in FIG. 6, schematic views of end to end anastomosis performed with the stapler of the present invention are illustrated. End of incised tract (i.e. intestine) and intestine's end is readily to reconnect to each other. Both ends are affixed to the trocar shaft (131) and an anvil (4) readily connected to the anvil shaft (41) respectively. When said trocar (5) fits in the clip notch (42) of said anvil shaft (41) in position, rotating the adjusting knob to close both tissues of intestine (c), after both tissues approach to each other within a reduced gap, said sound prompt (6) emits sound.

Said surgeon fires bolts according to the sound emission, said bolts pass through tissues of said intestine (c) into the anvil (4), once the bolts are bent and fixed to the anvil (4), the bent bolts are used to cleave said tissues of the intestine within circumferences of said bolts. After said tissues of intestine cleft, the in tact stapler can be pulled backward along the direction of the shaft (11) through said tract of the intestine (C). Said end of incised tract and said intestine's end are reconnected to form a straight passage in between, and the overall passage are formed with an inner opening throughout the passage and an overall radial closure around the cylindrical surface of the passage.

After the stapler is removed from the straight passage, an extra intestine tissue left within the stapler is discarded. Following surgery operations can be performed. Accordingly, the present invention is suitable for added pouched suture and cleaving and stapling of the tissues.

What is claimed is:

1. A circular stapler includes a bolting machine (1) consists of a shaft (11), an actuator (12) disposed to a proximal end of said shaft (11) and a stapler (13) disposed to a distal end of said shaft (11);
a hand-held handle (2) disposed to a bottom side of said actuator (12), when a safety catch (21) is released, operation of said handle can fire bolts from the stapler;
an adjusting knob (3) disposed to a proximal end of said actuator (12); and
an anvil (4) is connectable to said stapler (13), a hollow anvil shaft (41) is disposed to a proximal end of said anvil (4); characterized in that:
a conic trocar (5) disposed to a most distal end of said bolting machine (1), said trocar (5) fit in said anvil shaft (41); a cambered surface of said conic trocar (5) formed with, at least, three relieve-pressure grooves (53) equal distant distributed are extended from an apex (51) to a bottom (52);
an audio prompt (6) is disposed inside said bolting machine (1), said audio prompt (6) includes a printed circuit board (PCB) (61), a buzzer (62) electrically connected to the PCB (61), a switch (63) switches on/off said buzzer (62) and a battery (64) supplies power to the PCB (61).

2. The circular stapler of claim 1 wherein an included angle between each two adjacent relieve-pressure grooves (53) converged at the apex is 120 degrees.

3. The circular stapler of claim 1 wherein said relieve-pressure grooves (53) are concaved in said cylindrical surface and cambered in shape.

4. The circular stapler of claim 1 wherein a switch (63) disposed to a lateral face near said distal end of said bolting machine (1), where a firing indicator window showing a firing indicator scale formed (not shown).

5. The circular stapler of claim 1 wherein a proximal end of said anvil shaft (41) is formed with a clip notch (42).

6. The circular stapler of claim 1 wherein said safety catch (21) disposed between said bolting machine (1) and said handle (2) can be used to ensure an opportune firing of bolts.
